(19) United States
(12) Patent Application Publication
(10) Pub. No.: US 2011/0112439 A1
(43) Pub. Date: May 12, 2011

(54) Rotational Safety Syringe

(76) Inventors: Fu-Nan Chang, Taoyuan City (TW); Yen-Hung Liu, Taoyuan City (TW)

(21) Appl. No.: 12/614,137

(22) Filed: Nov. 6, 2009

(51) Int. Cl. A61B 5/151

(52) U.S. Cl. 600/583

(57) ABSTRACT

A rotational safety syringe includes front and rear barrels rotatable with respect to each other for moving a needle holder towards the rear barrel and compressing a spring to separate a protective cover. The needle holder is fixed by an elastic press button on the needle holder and a press opening on the rear barrel. After a through hole of the front barrel is aligned with a using position, the elastic press button can be pressed to release the needle holder, and the spring can eject the needle holder towards the through hole of the front barrel to extend the needle out from the through hole of the front barrel and pierce the needle into the using position. When the spring is released, the needle holder and needle are pulled back to hide the needle into the front and rear barrels to prevent the used needle from being touched.

### BACKGROUND OF THE INVENTION

(a) Field of the Invention

The present invention relates to a safety blood lancet, and more particularly to a rotational safety syringe whose syringe needle can be hidden automatically after use, to achieve the effects of providing a comprehensive safety to users and preventing a used syringe from being touched.

(b) Description of the Related Art

Ejective or intrusive blood collection devices are generally used in present medical examination procedures. For instance, medical professionals collect a blood sample from a diabetic patient by piercing a blood collection device into the patient's skin for controlling the patient's blood glucose concentration, dropping the collected blood sample onto a test strip, and inserting the test strip into a blood glucose meter to read a blood glucose concentration of the blood sample daily.

A blood lancet is generally pierced into a patient's skin and contacted with the patient's blood, and thus the patient may be infected easily by diseases (such as AIDS, Hepatitis B, and Hepatitis C, etc) through the blood lancet or syringe, and the tip of the needle of a conventional blood lancet, syringe or vacuum blood lancet is generally exposed to the outside after use, and thus a needle piercing injury may occur easily and endanger the safety of the medical professionals.

In view of the shortcomings of the conventional blood lancet and syringe description above, the inventor of the present invention based on years of experience in the related industry to conduct extensive researches and experiments, and finally developed a rotational safety syringe in accordance with the present invention to prevent a used needle from being touched or reused to assure the safety of medical professionals, users, and any other related people.

### SUMMARY OF THE INVENTION

Therefore, it is a primary objective of the present invention to overcome the shortcomings of the prior art by providing a rotational safety syringe capable of hiding a needle automatically after use, such that the used needle is prevented from being touched or causing piercing injuries and infections.

To achieve the foregoing objective, the invention provides a rotational safety syringe comprising a set of front and rear barrels, rotated with respect to each other, and having a needle holder and a spring, such that when the front and rear barrels are rotated, the needle holder is moved in an axial direction towards the rear barrel, while the spring is being pressed to fix the needle holder to the rear; and an elastic press button, being pressed for releasing the needle holder, and driving the spring to eject the needle holder instantly in a direction towards the through hole of the front barrel and extend the needle out from the through hole of the front barrel to pierce into a desired using position. If the spring is released to resume its position, the needle holder and the needle will be pulled back to hide the needle into the set of front and rear barrels, so as to prevent the used needle from being touched.

Another objective of the present invention is to install a protective cover for covering the needle of the needle holder, wherein the protective cover can be detached by moving the needle holder axially towards the rear barrel, and the protective cover provides a shielding effect to the needle and an easy way of distinguishing whether or not the needle has been used.

A further objective of the present invention is to hide the needle into the set of front and rear barrels automatically after the rotational safety syringe has been used. If the spring is released to return its original position, a protrusion of the needle holder remains at a predetermined path of the axial groove to restrict the rotation of the front and rear barrels, so as to prevent any intentionally reuse of the needle.

### BRIEF DESCRIPTION OF THE DRAWINGS

FIG. 1 is a perspective view of a rotational safety syringe of the present invention;

FIG. 2 is a cross-sectional view of a rotational safety syringe of the present invention;

FIG. 3 is a cross-sectional view of a rotational safety syringe with a protective cover attached in accordance with the present invention;

FIG. 4 is a cross-sectional view of a rotational safety syringe with a needle holder fixed in accordance with the present invention;

FIG. 5 is a schematic view of a pressed state of an elastic press button of a rotational safety syringe in accordance with the present invention;

FIG. 6 is a schematic view of an ejected status of a needle of a rotational safety syringe in accordance with the present invention;

FIG. 7 is a schematic view of a needle hidden in front and rear barrels of a rotational safety syringe in accordance with the present invention;

FIG. 8 is a cross-sectional view of a rotational safety syringe in accordance with another preferred embodiment of the present invention;

FIG. 9 is a perspective view of a rotational safety syringe in accordance with a further preferred embodiment of the present invention; and

FIG. 10 is a schematic view of a multi-sectional rotation status of an elastic press button of a rotational safety syringe in accordance with the present invention.

### DETAILED DESCRIPTION OF THE PREFERRED EMBODIMENTS

The technical characteristics of the present invention will become apparent with the detailed description of the preferred embodiments and the illustration of the related drawings as follows.

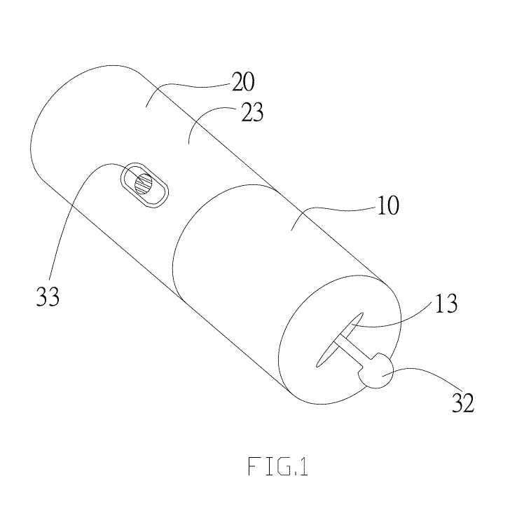

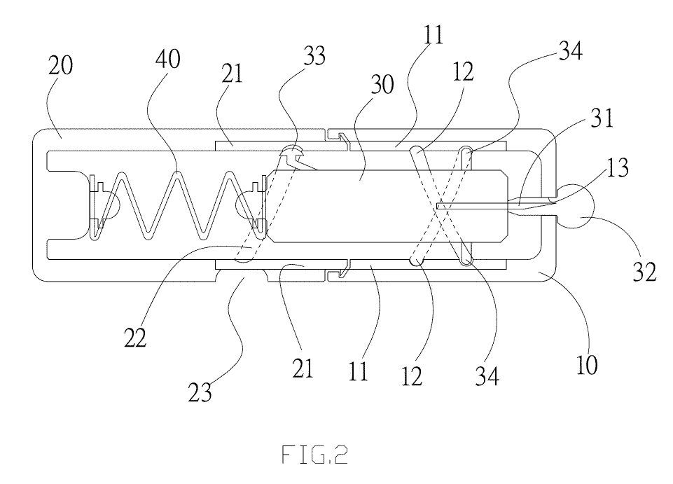

With reference to FIGS. 1 and 2 for a perspective view and a cross-sectional view of a rotational safety syringe of the present invention respectively, the rotational safety syringe comprises a set of a front barrel 10 and a rear barrel 20, a needle holder 30, and a spring 40.

The front barrel 10 and the rear barrel 20 are sheathed and rotated with respect to each other, and a through hole 13 is formed at a front end of the front barrel 10 for passing a needle 31 into or out of the through hole 13; at least one pair of corresponding engageable axial grooves 11, 21 and at least one pair of spiral grooves 12, 22 disposed on internal walls of the front and rear barrels 10, 20 respectively, wherein rear ends of the spiral grooves 12, 22 of the front and rear barrels 10, 20 are coupled to the axial grooves 11, 21 respectively, and a press opening 23 having a spiral penetrating cylindrical wall is formed at a joint between the spiral groove 22 and the axial groove 21 of the rear barrel 20.

The needle holder 30 is installed in the set of front and rear barrels 10, 20, and comprises a needle 31 installed at a front end of the needle holder 30, and a protective cover 32 covered onto the needle 31, wherein an end of the protective cover 32 extended from the through hole 13 of the front barrel 10 and out of the front barrel 10 has a width greater than the through hole 13, and an elastic press button 33 is installed on an external wall of the needle holder 30 for latching the press opening 23 and retracting the needle holder 30 into the spiral groove 22 and axial groove 21 of the rear barrel 20, and at least one protrusion 34 disposed on an external wall of the needle holder 30 for extending the needle holder 30 into the spiral groove 11 of the front barrel 10.

The spring 40 is installed in the set of front and rear barrels 10, 20 and between the rear barrel 20 and the needle holder 30, wherein two fixed ends of the spring 40 are fixed to the rear barrel 20 and the needle holder 30 respectively, such that when the spring 40 is not compressed, the needle 31 of the needle holder 30 is hidden into the set of front and rear barrels 10, 20.

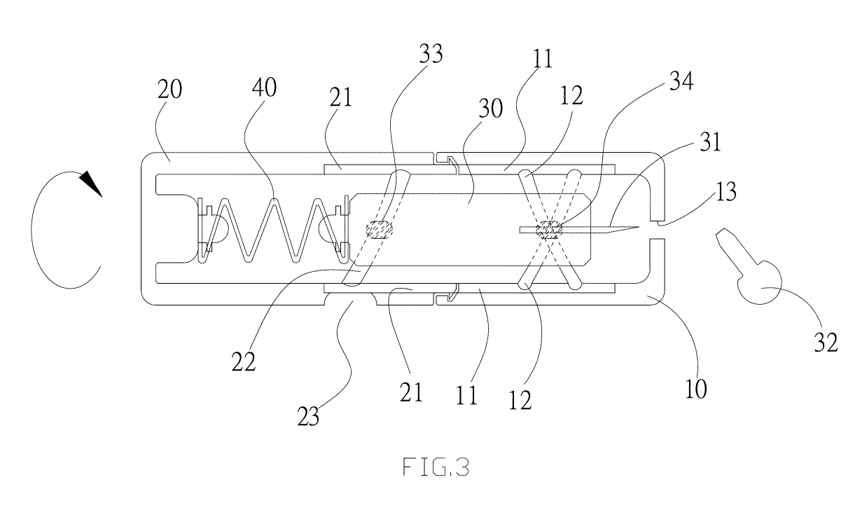

With reference to FIG. 3, the front and rear barrels 10, 20 are rotated with respect to each other when the rotational safety syringe of the present invention is used. With the guide of the protrusion 34 of the needle holder 30 and the spiral groove 12 of the front barrel 10, the needle holder 30 can be moved in an axial direction towards the rear barrel 20, while the spring 40 is being compressed to store energy for pushing the needle holder 30 towards the front barrel 10, such that when the needle holder 30 is moved in the direction towards the rear barrel 20, the protective cover 32 cannot pass through the through hole 13 and thus the protective cover 32 will be detached automatically.

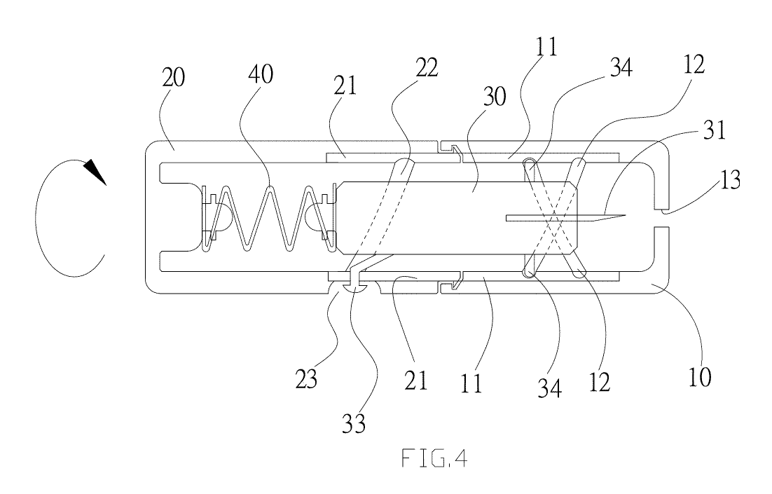

With reference to FIG. 4, the front and rear barrels 10, 20 are rotated with respect to each other. Until the elastic press button 33 reaches the joint between the spiral groove 22 and the axial groove 21, the elastic press button 33 constitute a latch effect with the press opening 23 to fix the needle holder 30 to the rear, and the elastic press button 33 and at least one protrusion 34 are aligned and engaged with the axial grooves 21, 11 of the rear barrel 20 and the front barrel 10 respectively.

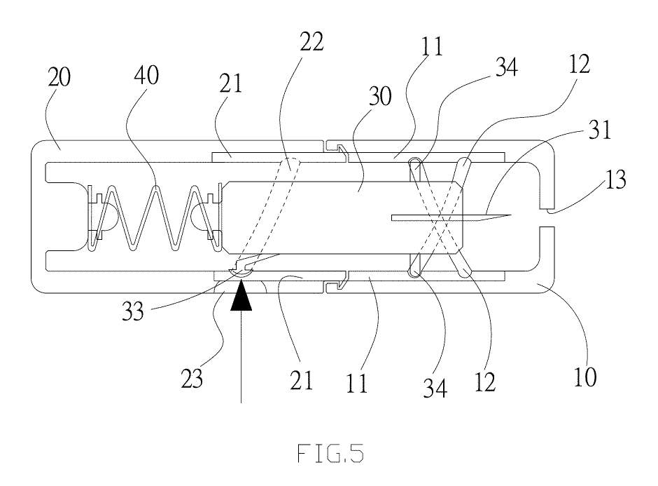

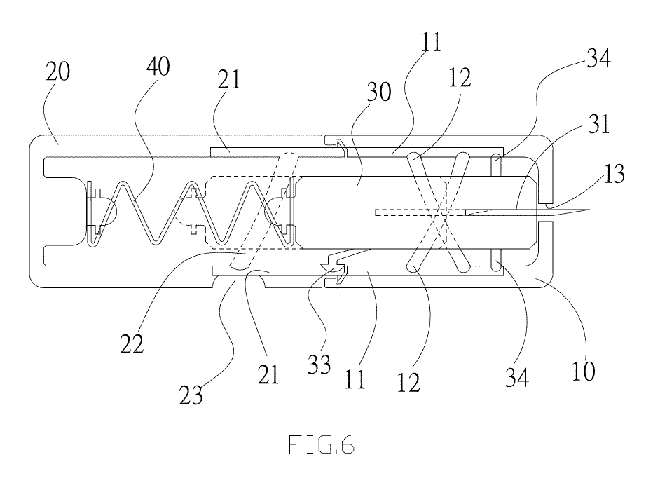

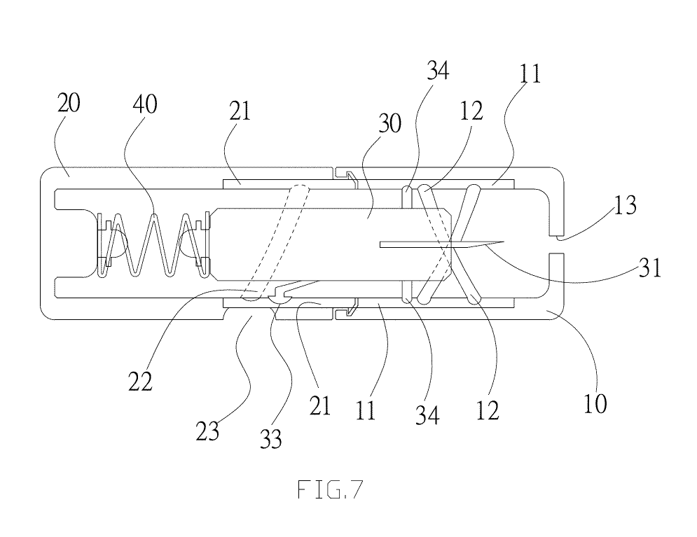

After the through hole 13 of the front barrel 10 is aligned with the using position, the elastic press button 33 can be pressed to release the needle holder 30 as shown in FIG. 5, such that the spring 40 rejects the needle holder 30 towards the through hole 13 of the front barrel 10 instantly as shown in FIG. 6, and the needle 31 is extended out from the through hole 13 of the front barrel 10 and pierced into the using position. When the spring 40 resumes its original uncompressed status as shown in FIG. 7, the needle holder 30 and the needle 31 are pulled back to hide the needle 31 into the set of front and rear barrels 10, 20, so as to prevent the used needle 31 from being touched.

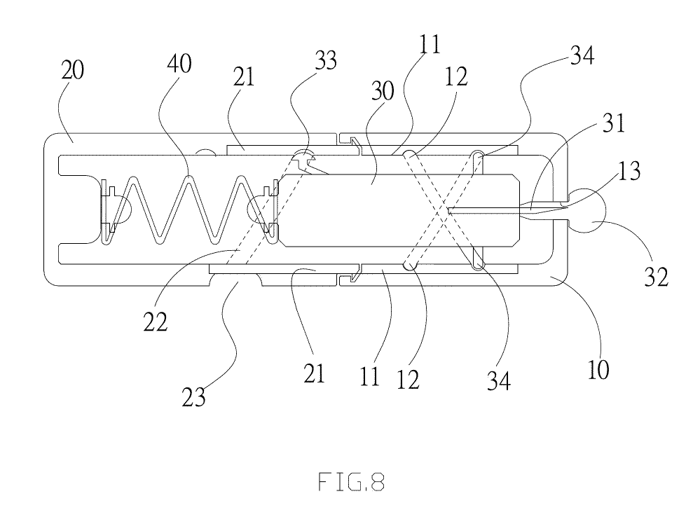

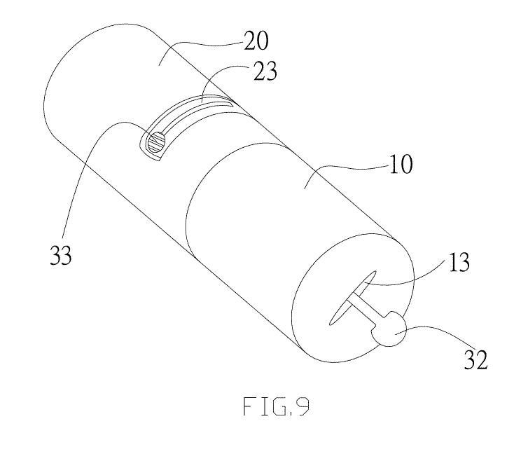

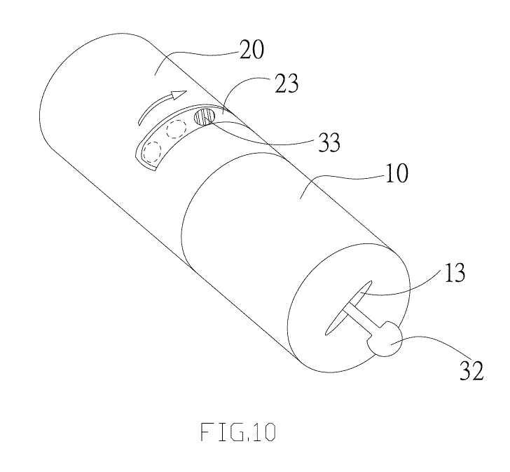

In addition, the distance between the spiral grooves in the front and rear barrels can be adjusted to change the overall elasticity and penetration force of the rotational safety syringe. As shown in FIG. 8, the distance between the spiral grooves 12, 22 is increased to extend the rotation path of the front and rear barrels 10, 20 and compress the spring 40 to produce larger compression elasticity, such that the needle 31 will provide larger penetration force during use. In addition, the press opening 23 can be a spiral groove with adjustable elasticity and depth as shown in FIGS. 9 and 10, and the path length of the spiral groove in the front and rear barrels can be increased. If it is necessary to adjust (such as increasing) the elasticity or penetration force, the front and rear barrels are rotated further with respect to each other to continue moving the elastic press button 33 along the press opening 23.

When the rotational safety syringe of the present invention is not in use as shown in FIG. 2, the protective cover 32 provides a shielding effect to the needle 31, and the protective cover 32 provides an easy way for users to quickly distinguish whether or not the syringe has been used. After the syringe has been used, the needle will be hidden into the set of front and rear barrels automatically. In FIG. 7, the spring 40 is situated at an uncompressed status, and thus the protrusion 34 of the needle holder 30 remains in a predetermined path of the axial groove 11 to restrict the front and rear barrels 10, 20 from rotating with respect to each other, so as to prevent an intentionally repeated use of the needle 31.

In summation of the description above, the present invention provides a better feasible rotational safety syringe that complies with the patent application requirements, and thus is duly filed for patent application. While the invention has been described by means of specific embodiments, numerous modifications and variations could be made thereto by those skilled in the art without departing from the scope and spirit of the invention set forth in the claims.

I claim:

1. A rotational safety syringe, comprising:
a set of a front barrel and a rear barrel, sheathed and rotated with respect to one another, and having at least one pair of oppositely engaged axial grooves formed on internal walls of the front and rear barrels respectively, and a rear end of at least one spiral groove of the front and rear barrels being coupled to the axial groove, and a press opening having a penetrating cylindrical wall and formed at a joint between the spiral groove and the axial groove of the rear barrel;
a needle holder, installed in the set of front and rear barrels, and including a needle installed at a front end of the needle holder, an elastic press button installed on an external wall of the needle holder for securing and holding the press opening, and the needle being retractable into the spiral groove and the axial groove, and at least one protrusion formed on an external wall of the needle holder for extending the needle holder into the spiral groove of the front barrel; and
a spring, installed into the set of front and rear barrels, and having two fixed ends fixed to the rear barrel and the needle holder respectively, such that when the spring is not compressed, the needle of the needle holder is hidden into the set of front and rear barrels.

2. The rotational safety syringe of claim 1, wherein the front barrel includes a through hole formed at a front end of the front barrel for passing the needle into and out of the through hole.

3. The rotational safety syringe of claim 1, wherein the front barrel includes a through hole formed at a front end of the front barrel for passing the needle into or out of the through hole, and the needle holder includes a protective cover covered onto the needle, and an end of the protective cover is extended out of the through hole of the front barrel, and the protective cover extended out from the front barrel has a width greater than the through hole.

4. The rotational safety syringe of claim 1, wherein the press opening is a spiral groove.

5. The rotational safety syringe of claim 1, wherein the distance between the spiral grooves in the front and rear barrels can be adjusted for changing an elasticity of the spring and a penetration force of the needle.
